(19) United States
(12) Patent Application Publication
(10) Pub. No.: US 2005/0070963 A1
(43) Pub. Date: Mar. 31, 2005

(54) Hair-removal apparatus for preparing a human torso for the use of an automated external defibrillator

(76) Inventors: Ellen Bartlett Wilson, Quincy, MA (US); Jean McTighe, Wellesley, MA (US)

Correspondence Address: GUERIN & RODRIGUEZ, LLP, 5 MOUNT ROYAL AVENUE, MOUNT ROYAL OFFICE PARK, MARLBOROUGH, MA, 01752, US

(21) Appl. No.: 10/674,959

(22) Filed: Sep. 30, 2003

(51) Int. Cl. A61N 1/18; A61N 1/22; A61N 1/28; A61N 1/36; A61N 1/368

(52) U.S. Cl. 607/5

(57) ABSTRACT

Described is a mechanism for removing hair from areas of a human torso in preparation for placing electrode pads of an automated external defibrillator on those areas. A preparation strip has a first pad portion, a second pad portion, and a center portion extending between the first and second pad portions. Dimensions of the strip enable one pad portion to be placed above the right breast while the other pad portion is placed below the left breast. The bottom surface of each pad portion has a contact region with a layer of adhesive effective to adhere to hair on the chest. When the preparation strip is attached to and subsequently separated from the torso, hair adhering to the adhesive layer of each contact region is removed, thus preparing those areas from which the hair is removed for placement of an electrode pad of an automated external defibrillator.

### FIELD OF THE INVENTION

The invention relates generally to automated external defibrillators, and more specifically, to preparation strips for removing hair from a human torso in preparation for using an automated external defibrillator.

### BACKGROUND

Hundreds of thousands of lives are lost each year to sudden cardiac arrest. Abnormal heart rhythms cause the majority of these deaths, with ventricular fibrillation being the most common cause. Ventricular fibrillation results from sudden erratic electrical impulses that prevent the heart muscles from working together to pump blood. Application of an electrical shock (defibrillation) stops the erratic impulses and allows coordinated rhythm and pumping to resume. Defibrillation is most effective when delivered within the first three to five minutes of the victim's collapse. If defibrillation is delayed by more than five minutes, the chance of survival is dramatically reduced and the possibility of experiencing permanent impairment of normal functions is significantly increased. Therefore, the ability to defibrillate quickly is crucial to improving survival rates and quality of life.

Defibrillators were initially developed for use by licensed physicians and trained emergency technicians in medical facilities with ready access to any drugs or additional equipment that might be required. This type of defibrillator allows the attending medical professional to manually adjust the size of the electrical charged based on the victim's condition and response. The development of lightweight, portable defibrillators that adjust the size of the electrical charge based on pre-programmed instructions instead of operator assessment (referred to as automated external defibrillators or AEDs) enable non-medical individuals to attempt defibrillation. As a result, AEDs make it possible to place defibrillators where individuals are most likely to be when a heart attack occurs: at home or in an office building, retail establishment, gated community, school, sports arena, theater, or health club. Placing defibrillators in public areas, in assisted living facilities, and in homes increases the possibility of getting the required aid to the victim within the critical three to five-minute period.

AEDs currently on the market include one- or two-piece electrode pads that are attached to the victim's chest by the individual administering aid. Once the leads between the AED unit and the electrodes are connected, the unit automatically begins to monitor the victim's condition and responds as programmed. The size of the electrical charge delivered by the unit is carefully controlled and, in contrast to the manual defibrillators used by medical professionals, cannot currently exceed 360 joules.

Because the size of the electrical charge delivered by an AED is limited; the electrode pads for AEDs must be applied directly to the victim's skin. Any significant chest hair on male victims must be removed prior to the application of the electrode pads, otherwise the hair can dissipate the limited electrical charge needed to defibrillate the heart. Accessory packs for all models of AEDs currently include a razor so the attendant can shave the victim's chest. In practice, however, razors pose certain difficulties. Because seconds count, shaving is an inefficient use of valuable time. Moreover, attendants in their haste may cut the victim's chest, causing bleeding and raising concerns with exposed blood. There remains, therefore, a need for a mechanism that can quickly remove a victim's chest hair in preparation for use with AEDs without the disadvantages of those aforementioned mechanisms.

### SUMMARY

In one aspect, the invention features an article of manufacture for use in removing hair from areas of a human torso in preparation for placing electrode pads of an automated external defibrillator on those areas. The article of manufacture comprises a preparation strip having a first pad portion, a second pad portion, and a center portion extending between the first and second pad portions. The preparation strip has dimensions that enable one of the pad portions to be placed above a right breast of the human torso while the other of the pad portions is placed below the left breast of the human torso. Each pad portion has a bottom surface that faces the human torso when the preparation strip is attached to the human torso. The bottom surface of each pad portion has a contact region with a layer of adhesive effective to adhere to hair on the human torso. When the preparation strip is attached to and subsequently separated from the human torso, hair adhering to the layer of adhesive of each contact region is removed from the human torso, thereby preparing those areas of the human torso from which the hair is removed for placement of an electrode pad of an automated external defibrillator.

In another aspect, the invention features an article of manufacture for use in removing hair from an area of a human torso in preparation for placing an electrode pad of an automated external defibrillator on that area. The article of manufacture comprises a preparation strip having a pad-portion. The pad portion has a surface with a contact region with dimensions sized to cover an area of skin when the preparation strip is attached to the human torso. The contact region has an adhesive effective for adhering to hair and causing the hair to be removed from the area of skin when the preparation strip is detached from the human torso. The area of skin from which hair is removed is sufficiently large to enable an electrode pad of an automated external defibrillator to be applied directly to the skin.

In another aspect, the invention features a method of removing hair from areas of a human torso in preparation for placing electrode pads of an automated external defibrillator on those areas. The method comprises adhering a pad portion of a preparation strip to the human torso. The pad portion has a contact region with an adhesive effective to adhere to hair on an area of skin on the human torso. The contact region has dimensions that approximate dimensions of an electrode pad of the automated external defibrillator. The preparation strip is separated from the human torso to remove hair adhering to the adhesive from the area of skin on the human torso, thereby preparing the area for the electrode pad of the automated external defibrillator to be applied directly to the skin.

### BRIEF DESCRIPTION OF THE DRAWINGS

The above and further advantages of this invention may be better understood by referring to the following description in conjunction with the accompanying drawings, in which like numerals indicate like structural elements and features in various figures. The drawings are not necessarily to scale, emphasis instead being placed upon illustrating the principles of the invention.

FIG. 1 is a plan view of the top surface of an embodiment of a one-piece preparation strip constructed in accordance with the principles of the invention.

FIG. 2 is a plan view of a bottom surface of the one-piece preparation strip of FIG. 1.

FIG. 3 is a diagram illustrating placement of the one-piece preparation strip of FIG. 1 on a human torso.

FIG. 4 is a plan view of the top surface of an embodiment of a “two-piece” preparation strip constructed in accordance with the principles of the invention.

FIG. 5 is a plan view of a bottom surface of the two-piece preparation strip of FIG. 4.

FIG. 6A is a plan view of another embodiment of a one-piece preparation strip constructed in accordance with the principles of the invention.

FIG. 6B is a plan view of an embodiment of a rectangular one-piece preparation strip constructed in accordance with the principles of the invention.

FIG. 6C is a plan view of another embodiment of a rectangular one-piece preparation strip constructed in accordance with the principles of the invention.

### DETAILED DESCRIPTION

The present invention features one-piece and two-piece adhesive preparation strips that can be used to quickly remove hair from the skin of a human torso in preparation for application of electrode pads from an automated external defibrillator or AED. Preparation strips of the invention can replace razors currently being included in AED unit accessory packets. With a preparation strip, hair removal is completed more quickly than with a razor. It also eliminates the risk of cuts and infection. In some embodiments, the preparation strip has easy-to-understand instructional information printed on the non-adhesive surface. Further, these markings on some embodiments of the preparation strip make it self-explanatory to use. From an examination of the preparation strip, an attendant can discern where to place the strip and the AED electrode pads on the human torso. Subsequent rapid removal of the preparation strip removes hair from certain areas of the torso and thus makes ready those areas for the placement of electrode pads connected to an AED. Yet another advantage is that disposal of the preparation strip, once used, is less hazardous than disposal of hazardous waste in the form of bloodstained articles that can be produced by use of the razor.

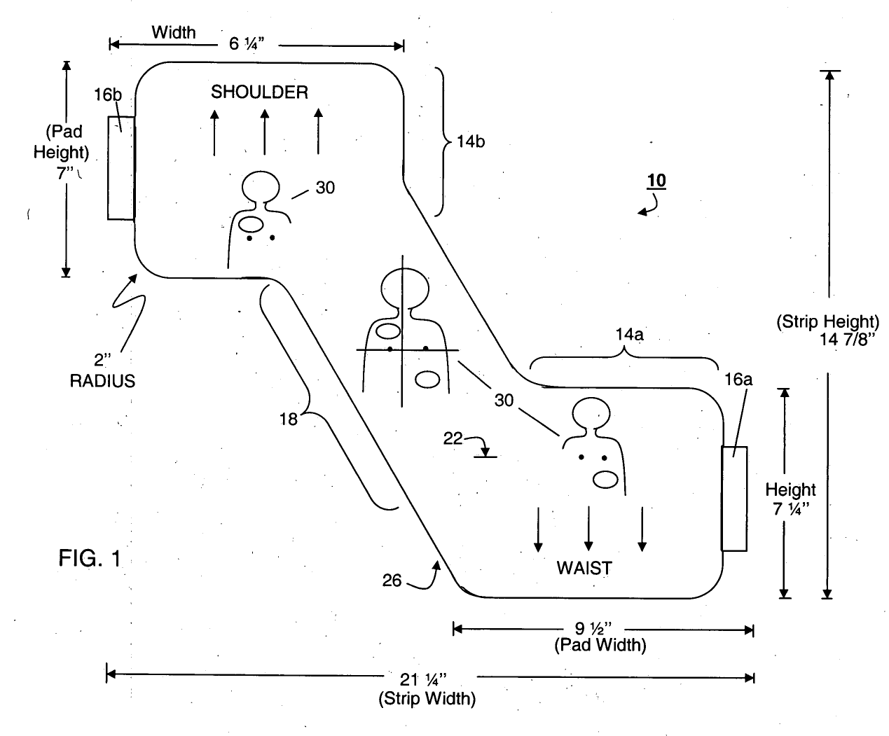

FIG. 1 shows an embodiment of a preparation strip 10 constructed in accordance with the principles of the invention. In one embodiment, the preparation strip 10 is constructed from conformable polyurethane, such as 3M cosmetic, non-woven material 9910, manufactured by 3M of St. Paul, Minn. The preparation strip 10 has a first pad portion 14a, a second pad portion 14b, and a center portion 18 extending between the pad portions 14a, 14b. In the embodiment shown, the pad portions 14a, 14b are generally rectangular in shape, and the corners of the pad portions 14a, 14b are rounded. In this embodiment, each pad portion 14a, 14b has a respective non-adhesive pull-tab 16a, 16b extending from an edge of the pad portion. Other embodiments of the preparation strip 10 have no pull-tabs, only one pull-tab (on only one of the two pad portions 14a, 14b), or more than one pull-tab for each pad portion. Pull-tabs can extend from a different edge of the pad portion 14a, 14b than that shown, from the non-adhesive top surface of the preparation strip 10, or from any combination thereof.

The preparation strip 10 also has a top surface 22 and a bottom surface 26. Displayed on the top surface 22 of this embodiment (e.g., printed, imprinted, raised print) is instructional information 30 for guiding the attendant in applying the preparation strip 10 to the torso of the human subject. Other embodiments of the preparation strip 10 do not display instructional information. For such embodiments, the shape of the preparation strip 10, alone or in combination with the location of adhesive, or instructions on the electrode pads or the AED unit, can be sufficient to guide the attendant as to where to place the strip on the human torso.

In the embodiment shown, the instructional information 30 appears on the top surface 22 of each of the portions 14a, 14b and 18, and includes diagrams that instruct the attendant regarding how to orient and place the preparation strip 10 on the subject's torso. At the center region 18, the instructional information 30 has a pad-placement diagram illustrating the relative positions of the pad portions 14a, 14b on a human torso. For each pad portion 14a, 14b, the instructional information 30 includes a pad-placement diagram, text description, and directional arrows. The pad-placement diagrams on each pad portion 14a, 14b diagrammatically show the location on the torso where that pad portion is to be placed. The text description identifies, by name, the particular feature of the human torso (e.g., waist, shoulder) near which that pad portion is to be placed. The directional arrows point generally in the direction of the particular feature, to assist the attendant in correctly orienting the preparation strip 10 on the subject's torso.

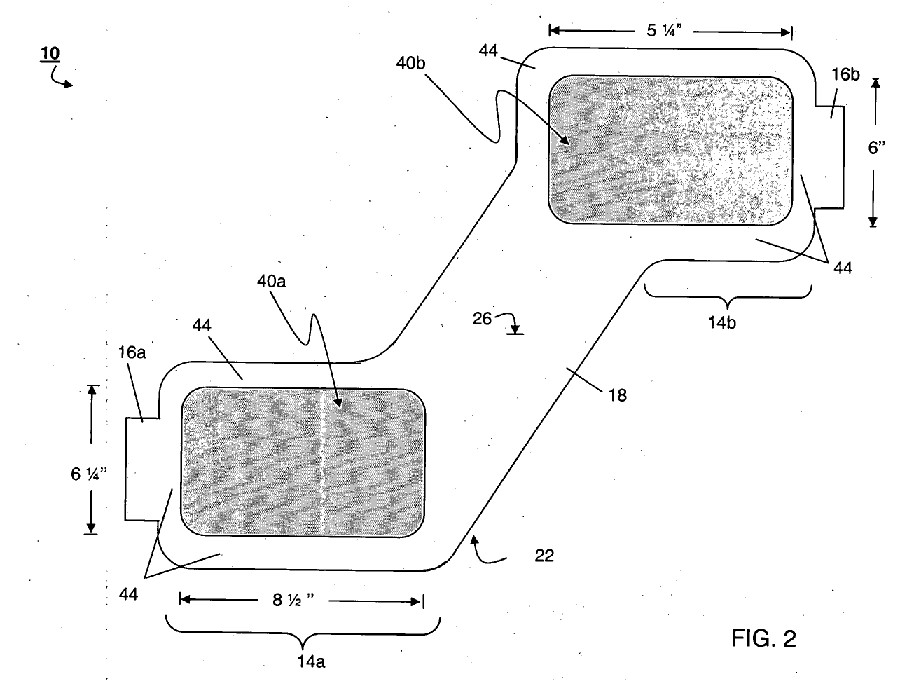

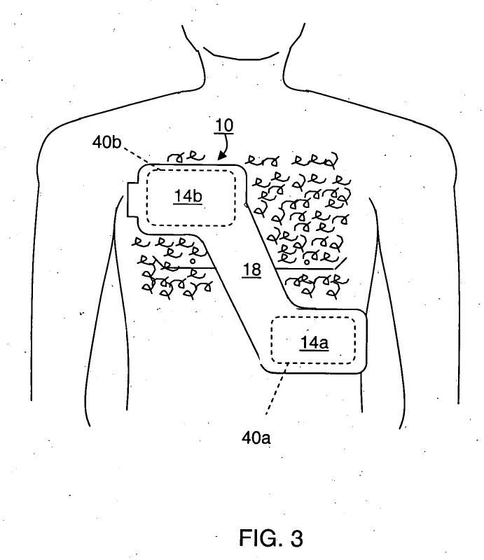

Also shown in FIG. 1 are exemplary dimensions for one embodiment of the preparation strip 10. The height and width of the preparation strip 10 are designed for an individual with a 42″ chest. Also, in this embodiment, the width of the pad portion 14a (which attaches near the waist) is longer than the width of the pad portion 14b (which attaches on the chest near the right shoulder). The additional width accommodates individuals with larger waists and permits part of the pad portion 14a to wrap around the side of the waist of thinner individuals, as shown in more detail in FIG. 3. In general, the size, shape, and orientation of each pad portion 14a, 14b are designed to correspond to the size, shape, and required orientation of the electrode pads of common commercially available AEDs. For example, the particular dimensions and pad orientation shown in FIG. 1 and in FIG. 2 (described below) accommodate the electrode pad of the portable ZOLL AED PLUS™, manufactured by ZOLL Medical Corporation of Burlington, Mass. The thickness of the preparation strip 10 (not shown) is approximately one-sixteenth of an inch. It should be recognized that the indicated dimensions, shape, and pad orientation refer to a certain embodiment of the preparation strip 10. The dimensions, shape, and orientation of the pad portions 14a, 14b and dimensions of the center portion 18 for other embodiments of the preparation strip 10 are designed to accommodate different sizes of human torsos, and different sizes, shapes, and placement of electrode pads for different types of AEDs or provided by different manufacturers, or combinations thereof.

FIG. 2 shows an embodiment of the bottom surface 26 of the preparation strip 10. Each pad portion 14a, 14b has a respective contact region 40a, 40b coated with a layer of adhesive. The adhesive on the contact regions 40a, 40b is effective for strongly adhering to hair such that the hair pulls from the skin when the preparation strip 10 is pulled from the torso. Preferably the adhesive acts as a depilatory to facilitate the removal of the hair, and is hypoallergenic to avoid any adverse allergic reaction or anaphylactic shock on the part of the human subject or the attendant. Also preferably, the adhesive leaves little or no residue that can interfere with the operation of the conductive gel used on the contact surface of an electrode pad of an AED. In one embodiment, the adhesive is an acrylate adhesive such as Adhesive No. 8083, manufactured by Medco Labs, Inc. of Bedford, Ohio.

When included in an AED unit, the preparation strip 10 may be enclosed (e.g., folded, sealed) in a package to keep the adhesive clean and effective. Protective release paper can be also placed over the adhesive layer. This covering keeps the preparation strip 10 from inadvertently sticking to itself or other items until the attendant positions the preparation strip 10 over the desired locations. In the embodiment shown, the contact regions 40a, 40b do not extend to the edge of the pad portions 14a, 14b. A non-adhesive grip edge 44 (e.g., ½″ wide) extends around a periphery of the contact regions 40a, 40b so that the attendant can more easily handle the preparation strip 10, for example, when removing the pads from the release paper or from the torso of the human subject. For other embodiments of the preparation strip 10, the grip edge is at a portion of the periphery of each contact region 40a, 40b (e.g., at one side of each contact region, instead of surrounding the contact regions 40a, 40b, as shown in FIG. 2). In another embodiment, the contact regions 40a, 40b extend to the edges of the pad portions 14a, 14b, but not onto the pull-tabs.

Exemplary dimensions for the contact regions 40a, 40b are shown. As shown, the contact region 40a for the pad portion 14a is wider than the contact region 40b of the pad portion 14b (the additional width corresponds to the wider pad portion 14a, described above). The dimensions of the contact regions 40a, 40b are designed to remove an area of hair sufficiently large to enable proper contact with an electrode pad of one or more commercially available AEDs. In one embodiment, these dimensions cover an area of skin on the human torso that is at least as large as an electrode pad of the automated external defibrillator. In another embodiment, the area of skin covered by the contact region 40a, 40b is slightly smaller than the size of the electrode pad, but large enough to ensure that an adequate portion of the electrode pad can be applied directly to the skin so that the automated external defibrillator can operate properly. The particular dimensions and orientations of the contact regions 40a, 40b for other embodiments of the preparation strip 10 are designed to accommodate different sizes, shapes, and placement of electrode pads for different AEDs produced by a variety of manufacturers.

Referring to FIG. 3, an attendant follows the instructional information 30 (FIG. 1) to apply the preparation strip 10 to the exposed torso of a human subject. The attendant places the pad portion 14b on the chest of the human subject, above the breast and below the right shoulder, and the pad portion 14a on the left side of the torso, below the breast and above the waist. A portion of the pad portion 14a can wrap around the side of the waist. The contact regions 40a, 40b of each respective pad portion 14a, 14b, as a result of the adhesive layer, hold fast to visible hair located on the torso depicted by the instructional information 30. The central portion 18 of the preparation strip 10 crosses from the upper right side to the lower left side of the torso and helps guide the attendant in obtaining proper separation and orientation of the adhesive contact regions 40a, 40b.

To apply and remove the preparation strip 10, the attendant is usually situated on one side of the supine subject. In this embodiment, the tabs 16a, 16b are on both sides of the subject, to make it easier for the attendant to get a firm grasp on the preparation strip 10 to apply it to and remove it from the subject. The attendant then rapidly pulls one of the pull-tabs 16a, 16b upwards to separate the preparation strip 10 from the skin. Hair adhering to the adhesive at each contact region 40a, 40b also separates from the torso with the preparation strip 10. Thus, this removal of the preparation strip 10 lays bare areas of skin corresponding to the size of the contact regions 40a, 40b.

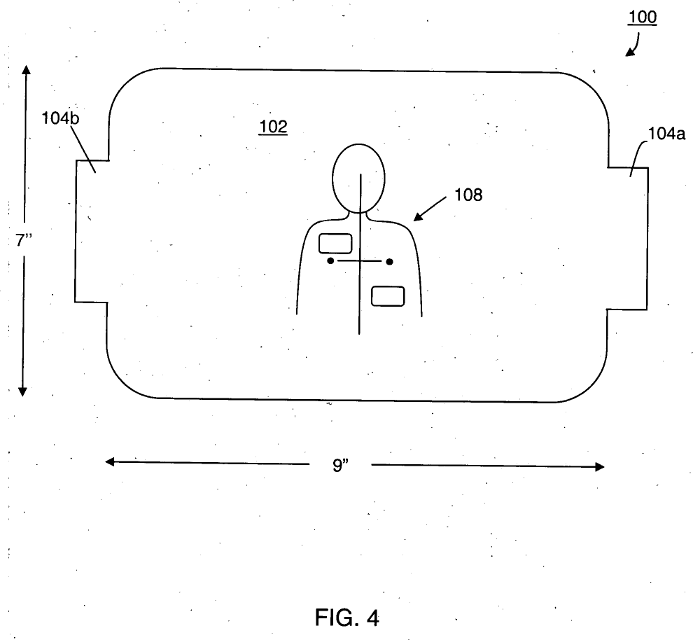

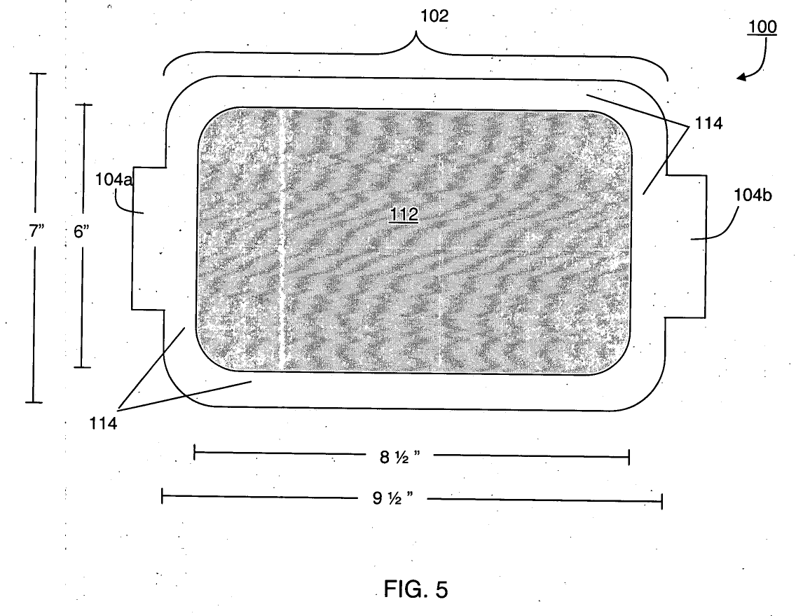

FIG. 4 and FIG. 5 illustrate an embodiment of a two-piece preparation strip 100 constructed in accordance with the principles of the invention. As used herein, two-piece means that two of such preparation strips 100 may be necessary to fully prepare a subject's torso for placement of the AED electrode pads, in contrast to the one-piece preparation strip 10 described above. FIG. 4 shows a top surface and FIG. 5 shows a bottom surface of the preparation strip 100. In this embodiment, the preparation strip 100 includes a pad portion 102 that is generally rectangular in shape with rounded corners. Other shapes of the pad portion can be used to practice the invention (e.g., circular, elliptical). At each end of the pad portion 102 is a non-adhesive pull-tab 104a, 104b. The pull-tabs 104a, 104b are on both sides of the pad portion 102, to make it easier for the attendant to get a firm grasp on the pad portion 102 after it has been applied to the torso. Other embodiments have no pull-tabs, only one pull-tab, or more than two pull-tabs. Pull-tabs can extend from different edges of the pad portion 102 than those shown, from the non-adhesive top surface of the preparation strip 100, or from any combinations thereof.

Instructional information 108 appears on the top surface of the preparation strip 100, diagrammatically illustrating where the preparation strip 100 is to be placed on the subject's torso in preparation for use of the AED. In situations where both preparation strips 100 are identical, the attendant can place the first preparation strip 100 at either location and a second preparation strip 100 at the other location. The diagram identifies one of two torso locations for applying the strip 100, one on the chest above the right breast and below the right shoulder, the other on the left side of the subject, below the left breast. Another embodiment of the preparation strip 100 does not have instructional information displayed on the top surface. Preparing a human torso for the use of an AED may not always require the use of two preparation strips 100. Occasionally, there is no hair present near the waist to interfere with the operation of the AED, and only one strip 100 is needed for removing hair from the chest.

The bottom surface of the pad portion 102 has a contact region 112 having an adhesive similar to that described above. The contact region 112 has dimensions that approximate the dimensions of an electrode pad of the automated-external defibrillator. When the preparation strip 100 is adhered to and subsequently separated from the torso, hair that adheres to the adhesive of the contact region 112 is removed from the torso. Thus, the affected area of the skin is prepared for attachment of the electrode pad of the automated external defibrillator.

In one embodiment, the dimensions of the contact region 112 are designed to cover an area of skin on the human torso that is at least as large as an electrode pad of the automated external defibrillator. In another embodiment, the area of skin covered by the contact region 112 is slightly smaller than the size of the electrode pad, but large enough to ensure that an adequate portion of the electrode pad can be applied directly to the skin so that the automated external defibrillator can operate properly.

In the embodiment shown, the contact region 112 does not extend to the edge of the pad portion 102. A non-adhesive grip edge 114 (e.g., approximately ½″ wide) extends around the periphery of the contact region 112 so that the attendant can more easily handle the preparation strip 100. Other embodiments of the preparation strip 100 include a grip edge at a portion of the periphery of the contact region 112 (e.g., at one side of the contact region, instead of surrounding the contact region 112 as shown in FIG. 5). In another embodiment, the contact region 112 extends to the edges of the pad portion 102, but not onto the pull-tabs 104a, 104b.

The shape and dimensions shown in FIG. 4 and in FIG. 5 are exemplary, and it should be recognized that the indicated dimensions refer to a certain embodiment of the preparation strip 100. The shape and dimensions of the preparation strip 100 can be varied to accommodate different sizes of human torsos, different sizes and shapes of electrode pads provided by different manufacturers or used for different types of AEDs, or combinations thereof. The various embodiments of the two-piece preparation strip 100 permit “mixing and matching,” that is, the two separate preparation strips 100 that are applied to the human subject can have the same or different dimensions for accommodating the electrode pads of the same or different manufacturers. For situations using two preparation strips 100 of different dimensions, the instructional information on each preparation strip 100 illustrates the appropriate placement of that preparation strip 100.

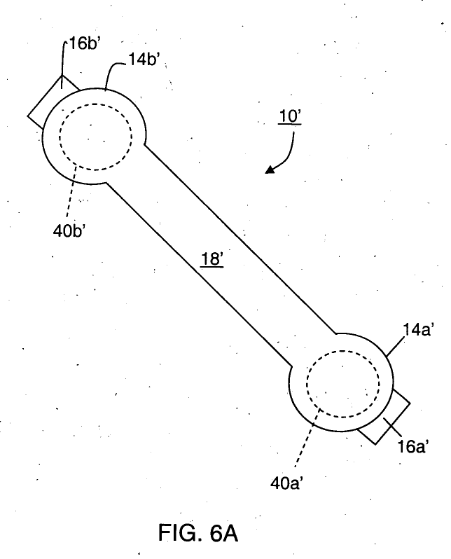

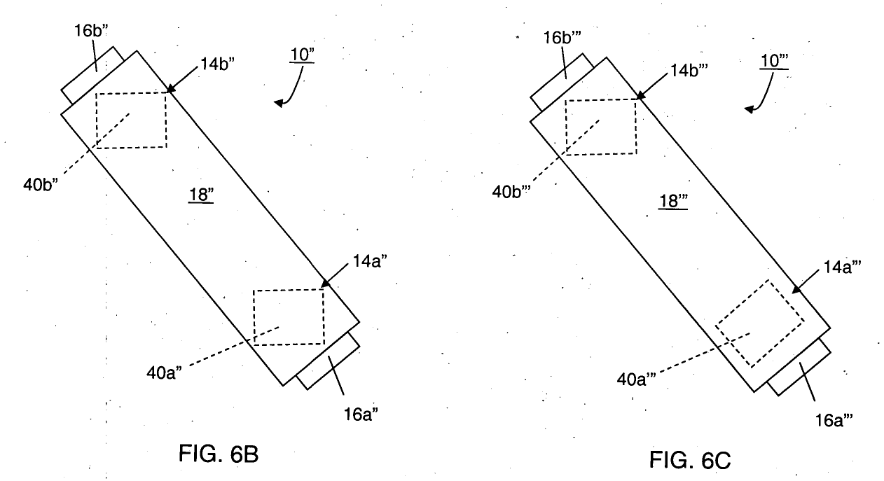

While the invention has been shown and described with reference to specific preferred embodiments, it should be understood by those skilled in the art that various changes in form and detail may be made therein without departing from the spirit and scope of the invention as defined by the following claims. For example, FIG. 6A, FIG. 6B, and FIG. 6C show alternative embodiments of preparation strips embodying the principles of the invention. FIG. 6A shows a barbell-shaped preparation strip 10′. FIG. 6B and FIG. 6C show rectangular-shaped preparation strips 10″ and 10′″ with different pad portion orientations. As shown in FIG. 6C, pad portions on a single preparation strip can have different orientations. Pad portions, center portions, contact regions, and tabs are shown with like reference numerals corresponding to like structural elements and features of the embodiments in FIG. 1, in FIG. 2, and in FIG. 3.

1. An article of manufacture for use in removing hair from areas of a human torso in preparation for placing electrode pads of an automated external defibrillator on those areas, the article of manufacture comprising a preparation strip having a first pad portion, a second pad portion, and a center portion extending between the first and second pad portions, the preparation strip having dimensions that enable one of the pad portions to be placed above a right breast of the human torso while the other of the pad portions is placed below the left breast, each pad portion having a bottom surface that faces the human torso when the preparation strip is attached to the human torso, the bottom surface of each pad portion having a contact region with a layer of adhesive effective to adhere to hair on the human torso so that when the preparation strip is attached to and subsequently separated from the human torso, hair adhering to the layer of adhesive of each contact region is removed from the human torso, thereby preparing those areas of the human torso from which the hair is removed for placement of an electrode pad of an automated external defibrillator.

2. The article of manufacture of claim 1, wherein each contact region has dimensions that approximate dimensions of an electrode pad of the automated external defibrillator.

3. The article of manufacture of claim 1, wherein the preparation strip further comprises a top surface having instructional information displayed thereon, the instructional information providing guidance for adhering the preparation strip to an appropriate area of the human torso.

4. The article of manufacture of claim 1, further comprising a pull-tab extending from an outside edge of one of the pad portions.

5. The article of manufacture of claim 1, further comprising a pull-tab extending from an outside edge of each pad portion.

6. The article of manufacture of claim 1, wherein the bottom surface has a non-adhesive grip edge at a portion of a periphery of the contact region of each pad portion.

7. The article of manufacture of claim 1, wherein the preparation strip is rectangular-shaped.

8. The article of manufacture of claim 1, wherein the preparation strip is barbell-shaped.

9. An article of manufacture for use in removing hair from an area of skin on a human torso in preparation for placing an electrode pad of an automated external defibrillator on that area of skin, the article of manufacture comprising a preparation strip having a pad portion, the pad portion having a surface with a contact region with dimensions sized to cover an area of skin when the preparation strip is attached to the human torso, the contact region having an adhesive effective for adhering to hair and for causing the hair to be removed from the area of skin when the preparation strip is detached from the human torso, the area of skin from which hair is removed being sufficiently large to enable an electrode pad of an automated external defibrillator to be applied directly to the skin.

10. The article of manufacture of claim 9, wherein the preparation strip further comprises a top surface having instructional information displayed thereon for providing guidance in adhering the preparation strip to an appropriate area of the human torso.

11. The article of manufacture of claim 9, further comprising a pull-tab extending from an edge of the pad portion.

12. The article of manufacture of claim 9, wherein the bottom surface has a non-adhesive grip edge at a portion of a periphery of the contact region.

13. A method of removing hair from areas of a human torso in preparation for placing electrode pads of an automated external defibrillator on those areas, the method comprising:
adhering a pad portion of a preparation strip to the human torso, the pad portion having a contact region with an adhesive effective to adhere to hair on an area of skin on the human torso, the contact region having dimensions that approximate dimensions of an electrode pad of the automated external defibrillator; and
separating the preparation strip from the human torso to remove hair adhering to the adhesive from the area of skin on the human torso, thereby preparing the area for the electrode pad of the automated external defibrillator to be applied directly to the skin.

14. The method of claim 13, further comprising providing instructional information on a top surface of the preparation strip to provide guidance in adhering the preparation strip to an appropriate area of the human torso.

15. The method of claim 13, wherein the preparation strip includes a second pad portion and a center portion extending between the pad portions, and further comprising adhering the second pad portion to the human torso.

16. The method of claim 15, wherein the steps of adhering pad portions to the human torso include placing one of the pad portions above the right breast of the human torso and the other of the pad portions below the left breast of the human torso.

17. The method of claim 13, further comprising adhering a pad portion of a second preparation strip to the human torso.

18. The method of claim 17, wherein the steps of adhering pad portions to the human torso include placing one of the pad portions above the right breast of the torso and the other of the pad portions below the left breast of the torso.

19. The method of claim 13, further comprising providing a non-adhesive grip edge at a portion of a, periphery of the contact region for facilitating grasping of the preparation strip by an attendant.

20. The method of claim 13, further comprising providing a pull-tab at an edge of the pad portion for facilitating grasping of the preparation strip by an attendant.
